(19) United States
(12) Patent Application Publication
(10) Pub. No.: US 2025/0191773 A1
(43) Pub. Date: Jun. 12, 2025

(54) SYSTEM AND METHOD OF PREDICTING A DISEASE RISK SCORE

(71) Applicant: OPEN DNA LTD., Haifa (IL)

(72) Inventors: Erez ORNAN, Haifa (IL); Eran FELDHAY, Tel Aviv (IL)

(21) Appl. No.: 18/969,458

(22) Filed: Dec. 5, 2024

Related U.S. Application Data
(60) Provisional application No. 63/606,626, filed on Dec. 6, 2023.

(51) Int. Cl. G16H 50/30; G16H 10/60; G16H 50/20

(52) U.S. Cl. CPC G16H 50/30; G16H 10/60; G16H 50/20

(57) ABSTRACT

A system and method of predicting a disease risk score of a target subject may include receiving a first feature dataset and a second feature dataset, comprising values of one or more features representing properties of the target subject. Embodiments may apply a first non-linear Machine Learning (ML) model on the first feature dataset, to obtain a first preliminary risk score, and applying a second non-linear ML based model on the second feature dataset, to obtain at least one respective, second preliminary risk score. Embodiments may select a subset of features from the first feature dataset and at least one second feature dataset, and subsequently applying a linear ML-based model on (i) the first preliminary risk score, (ii) the at least one second preliminary risk score, and (iii) the subset of features, to determine a disease risk score, representing an overall probability of the target subject in manifesting the disease.

### CROSS-REFERENCE TO RELATED APPLICATIONS

This application claims the benefit of priority of U.S. Patent Application No. 63/606,626, filed Dec. 6, 2023, which is hereby incorporated by reference in its entirety.

### FIELD OF THE INVENTION

The present invention relates generally to the technological field of assistive medical diagnosis. More specifically, the present invention relates to predicting a disease risk score.

### BACKGROUND OF THE INVENTION

The field of assistive medical diagnosis has seen significant advancements with the integration of data analytics. Traditional methods of predicting disease risk often rely on limited datasets and models, which may not capture the complex interactions between various risk factors. These methods can result in less accurate predictions and generalized treatment recommendations.

Existing solutions face challenges in integrating diverse data types, such as genomic, clinical, and lifestyle data, to provide personalized risk assessments. The need for a more comprehensive approach that leverages advanced techniques to analyze multifaceted datasets is evident. This approach can enhance the accuracy of disease risk predictions and enable tailored healthcare recommendations, addressing the limitations of current methodologies.

Machine-learning based assistive diagnosis and risk assessment have emerged as promising solutions. However, these approaches also have their downsides. The first approach involves using linear AI models. While linear models are simpler and more interpretable, they may fail to capture the complex relationships between patient features and the desired prediction, such as disease risk. This limitation can lead to less accurate predictions and suboptimal treatment recommendations.

The second approach involves using complex, non-linear AI models. These models, such as deep learning neural networks, can capture intricate and non-linear relationships between various patient features and the disease risk. However, the downside of this approach is that it often lacks interpretability. The complex nature of these models makes it challenging to generate intuitive and clear recommendations for healthcare providers and patients. The lack of transparency in how the model arrives at its predictions can hinder the trust and adoption of such systems in clinical practice.

### SUMMARY OF THE INVENTION

Therefore, there is a need for a system that combines the strengths of both approaches. Such a system should be capable of capturing complex relationships between patient features and disease risk while maintaining a level of interpretability that allows for clear and actionable recommendations. This hybrid approach can provide more accurate and personalized risk assessments, ultimately improving patient outcomes and advancing the field of assistive medical diagnosis.

Embodiments of the invention may include a method and system for determining a disease risk score, by at least one processor. Embodiments of the method may include receiving, for each of a plurality of subjects (e.g., patients) information that includes, for example, and/or genomic data. Each subject may have a specified type of complex polygenic disease risk. The at least one processor may use a Machine Learning (ML) based model to determine the subject's disease risk, based on the received data.

The datasets for training the machine learning model can include genomic and pharmacogenomic data such as polygenic or monogenic data as well as clinical data such as history, medications, biomarkers and lifestyle data. A machine learning model can use different datasets with different data elements for each subject.

The training may be done using ensemble learning, by running training over multiple datasets and using multiple training cycles. In some embodiments these include additional training over negative cases. In some embodiments different datasets contain different data elements. The final stage of training may include combining the predictions from training over multiple datasets and cycles into final predictions.

At an inference stage, embodiments of the invention may apply the trained ensemble learning model to data pertaining to a specific target subject. Embodiments of the invention may aggregate the scores received with respect to a target subject, to predict a risk of the subject to at least one of the diseases in a set of diseases, and provide best treatment recommendations for the individual subject.

Embodiments of the invention may include a user interface that may allow manipulation of risk factor data elements by a user, to visualize the effect of changes in risk factor values on the subject's risk level.

Based on the calculated risk level of an individual and various other parameters, treatment options and therapies may be suggested according to pre-defined risk levels, machine learning models or pharmacogenomic data.

Embodiments of the invention may offer a unique approach to assessing the risk of various health conditions by integrating genetic and clinical data, enabling accurate risk predictions and personalized healthcare recommendations.

A dataset including all available features may be built, and can include polygenic scores, monogenic data, pharmacogenomic data, age, sex, race and ethnicity, genetic scores (from stage 1), patient medical history, smoking habits, total cholesterol, low-density and high density lipoprotein cholesterol, systolic blood pressure, diabetes, use of medications, blood biochemistry measurements, physical activity related parameters and mental health related parameters serves as input for dataset creation. Missing values can be input. Multiple data sources can be used, and multiple subsets of data can be used.

Embodiments of the invention may perform feature selection, to select the set of features. Various machine learning classifiers may be checked, and the best performing classifier may be selected.

Gender specific datasets (separate male and female datasets) may be created using a similar process, missing values are imputed, and feature selection is performed to select the set of features.

Various machine learning classifiers may be checked, and the best performing gender specific classifiers may be selected.

In some embodiments, the best performing ML model (base model) nay be used for initial prediction, then the negative predictions from the base model may serve as inputs for subsequent prediction using the best performing gender-specific models, positives from either the base model or from the subsequent gender-specific models are marked as positives in the final result set, and negatives from the gender-specific models are marked as negatives in the final results set.

Performance may be checked on a separate validation set. Additionally, or alternatively, multiple datasets may include different data elements (features) and predictions from multiple models may be aggregated into final results e.g. using voting.

The user interface may show the results of the algorithm to the user. It may show risk levels and recommended treatment options. It may allow manipulation of the algorithm parameters to see the effect on the risk level and treatment recommendations.

Embodiments of the invention may include a method and system for determining a disease risk score, e.g., a probability of a target subject in manifesting a disease, by at least one processor.

According to some embodiments, the at least one processor may receive a first feature dataset and at least one second feature dataset. Each feature dataset may include values of one or more features, and each feature may represent a property of the target subject.

The at least one processor may apply a first non-linear Machine Learning (ML) based model on the first feature dataset, to obtain a first preliminary risk score, representing a first assessed probability of the target subject in manifesting the disease, e.g., a risk of expressing the disease within a predetermined time period (e.g., 10 years), based on the first feature dataset.

Additionally, the at least one processor may apply at least one second non-linear ML based model on respective feature dataset of the at least one second feature dataset, to obtain at least one respective, second preliminary risk score. The second preliminary risk score may represent a second assessed probability of the target subject in manifesting the disease, based on the at least one second feature dataset.

According to some embodiments, the at least one processor may select a subset of features from the first feature dataset and at least one second feature dataset, and apply a linear ML-based model on (i) the first preliminary risk score, (ii) the at least one second preliminary risk score, and (iii) the subset of features, to determine a disease risk score that represents an overall probability of the target subject in manifesting the disease (e.g., within the predetermined time period).

According to some embodiments, the first feature dataset and the at least one second feature dataset may overlap in one or more features.

The first feature dataset may include values of genomic features, selected from a list consisting of: a Polygenic Risk Score (PRS), representing polygenic risk in manifesting the disease, monogenic data, representing traits of the target subject that are influenced by a single gene, and pharmacogenomic data, representing the target subject's response to drugs.

The first non-linear ML model may include a Deep Learning Neural Network (DNN), having at least four neural layers, adapted to learn complex, and optionally non-linear relations between features of the first feature dataset and the first preliminary risk score.

According to some embodiments, the at least one second feature dataset may include values of features pertaining to a first group of blood measurements, selected from: a blood pressure (BP) level, a total cholesterol level, a High Density Lipoprotein (HDL) level, a Low Density Lipoprotein (LDL) level, a Lipoprotein A level.

Additionally, or alternatively, the at least one second feature dataset may include values of features pertaining to a second group of blood measurements, selected from: a testosterone level, a C-reactive protein level, a basophil count, a cystatin-C level, and a mean corpuscular hemoglobin value.

Additionally, or alternatively, the at least one second feature dataset may include values of features of the subject, selected from a list consisting of: an age, a gender, an ethnicity, a prior diagnosis, a status of drug treatment, a lifestyle factor, a feature or indication related to mental health, and the like.

The at least one second non-linear ML model may be selected from a list of ML based architectures, such as a decision tree model, a k-Nearest Neighbors model, a non-linear Support Vector Machine (SVM) model, a Naive Bayes model, having non-linear transformations, and the like. The at least one second non-linear ML model may be adapted to learn complex, and optionally non-linear relations between features of the first feature dataset and the first preliminary risk score.

According to some embodiments, the at least one processor may receive (e.g., during a training stage) a first training dataset. The first training dataset may include a plurality of first feature datasets, each pertaining to a respective subject of a first cohort of subjects. The at least one processor may further obtain a first set of annotations, each labeling a condition of a corresponding subject of the first cohort of subjects. The at least one processor may use the first set of annotations as supervisory data, to train the first non-linear ML model, so as to predict the first preliminary risk score of subjects of the first cohort of subjects, based on the first training dataset.

Additionally, or alternatively, the at least one processor may receive a second training dataset. The second training dataset may include a plurality of second feature datasets, each pertaining to a respective subject of a second cohort of subjects, which may, or may not overlap with the first cohort of subjects. The at least one processor may further obtain a second set of annotations, each labeling a condition of a corresponding subject of the second cohort of subjects. The at least one processor may use the second set of annotations as supervisory data, to train a second non-linear ML model of the at least one second non-linear ML models, so as to predict the second preliminary risk score of subjects of the second cohort of subjects, based on the second training dataset.

According to some embodiments, the at least one processor may select the subset of features from the first dataset and/or second dataset, wherein the subset of features pertains to a specific subject of the first cohort and/or second cohort. The at least one processor may use at least one of (i) the first set of annotations and (ii) the second set of annotations as supervisory data, to train the linear ML-based model to produce an initial prediction of a disease risk score of the specific subject, wherein the initial prediction may a linear combination of the subset of features of the specific subject, and (a) the first preliminary risk score of the specific subject and/or (b) the at least one second preliminary risk score of the specific subject.

According to some embodiments, the at least one processor may employ a feature selection algorithm, to identify a first group of features from the first feature dataset, as prominent contributors in predicting the first preliminary risk score. The at least one processor may further employ the feature selection algorithm, to identify a second group of features from the second feature dataset, as prominent contributors in predicting the second preliminary risk score. The at least one processor may subsequently select the subset of features of the plurality of features based on the first, and second groups of features.

According to some embodiments, the at least one processor may calculate one or more disease-specific statistical properties, characterizing a manifestation of the disease in a population of the first cohort and/or second cohort, based on at least one of the first and second sets of annotations.

For example, the at least one processor may calculate a mean and standard deviation of probability of manifestation of the disease of interest in various subsets of the population, e.g., based on ethnicity, age, gender, and the like. The at least one processor may obtain, from the linear ML-based model, an initial value of the disease risk score for the target subject. It may be appreciated that this initial disease risk value may be biased, in a sense that it may relate to a general population (e.g., the first and/or second subject cohorts), and disregard the characteristics of the population's subset, to which the specific subject pertains.

The at least one processor may amend this bias, by applying statistical methods (e.g., normalization, calibration, post-stratification, etc.) as known in the art, so as to fine-tune the initial value of the disease risk score, based on the disease-specific statistical properties, and to determine a final disease risk score of the target subject.

According to some embodiments, the at least one processor may employ a Graphical User Interface (GUI), and may present the final, predicted disease risk score via the GUI.

Additionally, or alternatively, the at least one processor may receive, via the GUI, a perturbation, or change in a value of at least one feature of the subset of features. The at least one processor may apply the linear ML-based model on the subset of features having the perturbed feature value, to determine a simulated disease risk score, representing a simulated probability of the target subject in manifesting the disease. The at least one processor may proceed to present the simulated disease risk score via the GUI as a result of that perturbation.

Additionally, or alternatively, the at least one processor may employ a generative model, adapted to produce a recommendation for improving the predicted, final disease risk score. Such a recommendation may include a suggested change in one or more values of the subset of features, and may be adjoint with a predicted change, or improvement of the calculated disease risk score.

For example, the at least one processor may automatically perturb a value of one or more features of the subset of features. These perturbations may include, for example: a change in treatment (such as administering a drug, or a change in a dosage thereof, and the like), a change of lifestyle (such as enhancement of physical activity, cessation of smoking, etc.), or an effect of a change in any physical or chemical biomarker (e.g., a change in blood pressure, a change in cholesterol levels, etc.).

The at least one processor may then apply the linear ML-based model on the subsets of features, each having at least one perturbed feature value, to determine corresponding simulated disease risk scores. Each simulated disease risk score may represent a simulated probability of the target subject in manifesting the disease, as a result of the corresponding at least one perturbation. The at least one processor may proceed to select a preferred perturbation, that may be associated with a maximal improvement (e.g., decrease) in the calculated disease risk score. The at least one processor may present the simulated disease risk scores via the GUI, as recommendations for diminishing the target subject's probability of manifesting the disease.

Embodiments of the invention may include a system for predicting a disease risk score of a subject (e.g., a patient). Embodiments of the system may include a non-transitory memory device, wherein modules of instruction code are stored, and at least one processor associated with the memory device, and configured to execute the modules of instruction code.

Upon execution of said modules of instruction code, the at least one processor may be configured to: receive a first feature dataset and at least one second feature dataset, wherein each feature dataset comprises values of one or more features, and wherein each feature represents a property of the target subject; apply a first non-linear ML based model on the first feature dataset, to obtain a first preliminary risk score, representing a first assessed probability of the target subject in manifesting the disease; apply a second non-linear ML based model on the at least one second feature dataset, to obtain at least one respective, second preliminary risk score, representing a second assessed probability of the target subject in manifesting the disease; select a subset of features from the first feature dataset and at least one second feature dataset; and apply a linear ML-based model on (i) the first preliminary risk score, (ii) the at least one second preliminary risk score, and (iii) the subset of features, to determine a disease risk score, representing an overall probability of the target subject in manifesting the disease.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1 is a block diagram, depicting a computing device which may be included in a system for determining a disease risk score, according to some embodiments of the invention;

FIG. 2 is a block diagram, depicting a system for determining a disease risk score, according to some embodiments of the invention;

FIGS. 3A and 3B are screenshots of a Graphic User Interface (GUI), which may be included in an implementation of embodiments of the invention;

FIG. 4 is a flow diagram, depicting stages of a method of determining a disease risk score, according to some embodiments of the invention.

It will be appreciated that for simplicity and clarity of illustration, elements shown in the figures have not necessarily been drawn to scale. For example, the dimensions of some of the elements may be exaggerated relative to other elements for clarity. Further, where considered appropriate, reference numerals may be repeated among the figures to indicate corresponding or analogous elements.

### DETAILED DESCRIPTION OF THE PRESENT INVENTION

One skilled in the art will realize the invention may be embodied in other specific forms without departing from the spirit or essential characteristics thereof. The foregoing embodiments are therefore to be considered in all respects illustrative rather than limiting of the invention described herein. Scope of the invention is thus indicated by the appended claims, rather than by the foregoing description, and all changes that come within the meaning and range of equivalency of the claims are therefore intended to be embraced therein.

In the following detailed description, numerous specific details are set forth in order to provide a thorough understanding of the invention. However, it will be understood by those skilled in the art that the present invention may be practiced without these specific details. In other instances, well-known methods, procedures, and components have not been described in detail so as not to obscure the present invention. Some features or elements described with respect to one embodiment may be combined with features or elements described with respect to other embodiments. For the sake of clarity, discussion of same or similar features or elements may not be repeated.

Although embodiments of the invention are not limited in this regard, discussions utilizing terms such as, for example, “processing,” “computing,” “calculating,” “determining”, “establishing”, “analyzing”, “checking”, or the like, may refer to operation(s) and/or process(es) of a computer, a computing platform, a computing system, or other electronic computing device, that manipulates and/or transforms data represented as physical (e.g., electronic) quantities within the computer's registers and/or memories into other data similarly represented as physical quantities within the computer's registers and/or memories or other information non-transitory storage medium that may store instructions to perform operations and/or processes.

Although embodiments of the invention are not limited in this regard, the terms “plurality” and “a plurality” as used herein may include, for example, “multiple” or “two or more”. The terms “plurality” or “a plurality” may be used throughout the specification to describe two or more components, devices, elements, units, parameters, or the like. The term “set” when used herein may include one or more items.

Unless explicitly stated, the method embodiments described herein are not constrained to particular order or sequence. Additionally, some of the described method embodiments or elements thereof can occur or be performed simultaneously, at the same point in time, or concurrently.

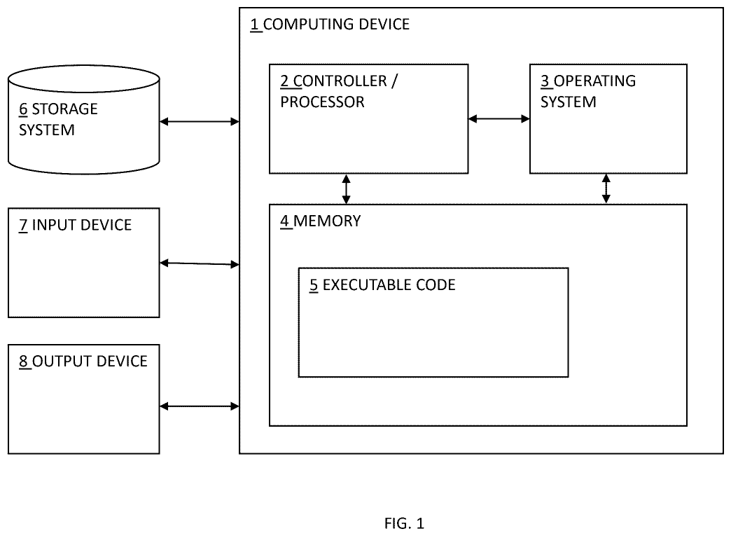

Reference is now made to FIG. 1, which is a block diagram depicting a computing device, which may be included within an embodiment of a system for predicting a disease risk score, according to some embodiments.

Computing device 1 may include a processor or controller 2 that may be, for example, a central processing unit (CPU) processor, a chip or any suitable computing or computational device, an operating system 3, a memory 4, executable code 5, a storage system 6, input devices 7 and output devices 8. Processor 2 (or one or more controllers or processors, possibly across multiple units or devices) may be configured to carry out methods described herein, and/or to execute or act as the various modules, units, etc. More than one computing device 1 may be included in, and one or more computing devices 1 may act as the components of, a system according to embodiments of the invention.

Operating system 3 may be or may include any code segment (e.g., one similar to executable code 5 described herein) designed and/or configured to perform tasks involving coordination, scheduling, arbitration, supervising, controlling or otherwise managing operation of computing device 1, for example, scheduling execution of software programs or tasks or enabling software programs or other modules or units to communicate. Operating system 3 may be a commercial operating system. It will be noted that an operating system 3 may be an optional component, e.g., in some embodiments, a system may include a computing device that does not require or include an operating system 3.

Memory 4 may be or may include, for example, a Random-Access Memory (RAM), a read only memory (ROM), a Dynamic RAM (DRAM), a Synchronous DRAM (SD-RAM), a double data rate (DDR) memory chip, a Flash memory, a volatile memory, a non-volatile memory, a cache memory, a buffer, a short term memory unit, a long term memory unit, or other suitable memory units or storage units. Memory 4 may be or may include a plurality of possibly different memory units. Memory 4 may be a computer or processor non-transitory readable medium, or a computer non-transitory storage medium, e.g., a RAM. In one embodiment, a non-transitory storage medium such as memory 4, a hard disk drive, another storage device, etc. may store instructions or code which when executed by a processor may cause the processor to carry out methods as described herein.

Executable code 5 may be any executable code, e.g., an application, a program, a process, task, or script. Executable code 5 may be executed by processor or controller 2 possibly under control of operating system 3. For example, executable code 5 may be an application that may predict a disease risk score, as further described herein. Although, for the sake of clarity, a single item of executable code 5 is shown in FIG. 1, a system according to some embodiments of the invention may include a plurality of executable code segments similar to executable code 5 that may be loaded into memory 4 and cause processor 2 to carry out methods described herein.

Storage system 6 may be or may include, for example, a flash memory as known in the art, a memory that is internal to, or embedded in, a micro controller or chip as known in the art, a hard disk drive, a CD-Recordable (CD-R) drive, a Blu-ray disk (BD), a universal serial bus (USB) device or other suitable removable and/or fixed storage unit. Data pertaining to a specific subject (e.g., patient) may be kept in storage system 6 and may be loaded from storage system 6 into memory 4 where it may be processed by processor or controller 2. In some embodiments, some of the components shown in FIG. 1 may be omitted. For example, memory 4 may be a non-volatile memory having the storage capacity of storage system 6. Accordingly, although shown as a separate component, storage system 6 may be embedded or included in memory 4.

Input devices 7 may be or may include any suitable input devices, components, or systems, e.g., a detachable keyboard or keypad, a mouse and the like. Output devices 8 may include one or more (possibly detachable) displays or monitors, speakers and/or any other suitable output devices. Any applicable input/output (I/O) devices may be connected to Computing device 1 as shown by blocks 7 and 8. For example, a wired or wireless network interface card (NIC), a universal serial bus (USB) device or external hard drive may be included in input devices 7 and/or output devices 8. It will be recognized that any suitable number of input devices 7 and output device 8 may be operatively connected to Computing device 1 as shown by blocks 7 and 8.

A system according to some embodiments of the invention may include components such as, but not limited to, a plurality of central processing units (CPU) or any other suitable multi-purpose or specific processors or controllers (e.g., similar to element 2), a plurality of input units, a plurality of output units, a plurality of memory units, and a plurality of storage units.

The term neural network (NN) or artificial neural network (ANN), e.g., a neural network implementing a machine learning (ML) or artificial intelligence (AI) function, may be used herein to refer to an information processing paradigm that may include nodes, referred to as neurons, organized into layers, with links between the neurons. The links may transfer signals between neurons and may be associated with weights. A NN may be configured or trained for a specific task, e.g., pattern recognition or classification. Training a NN for the specific task may involve adjusting these weights based on examples. Each neuron of an intermediate or last layer may receive an input signal, e.g., a weighted sum of output signals from other neurons, and may process the input signal using a linear or nonlinear function (e.g., an activation function). The results of the input and intermediate layers may be transferred to other neurons and the results of the output layer may be provided as the output of the NN. Typically, the neurons and links within a NN are represented by mathematical constructs, such as activation functions and matrices of data elements and weights. At least one processor (e.g., processor 2 of FIG. 1) such as one or more CPUs or graphics processing units (GPUs), or a dedicated hardware device may perform the relevant calculations.

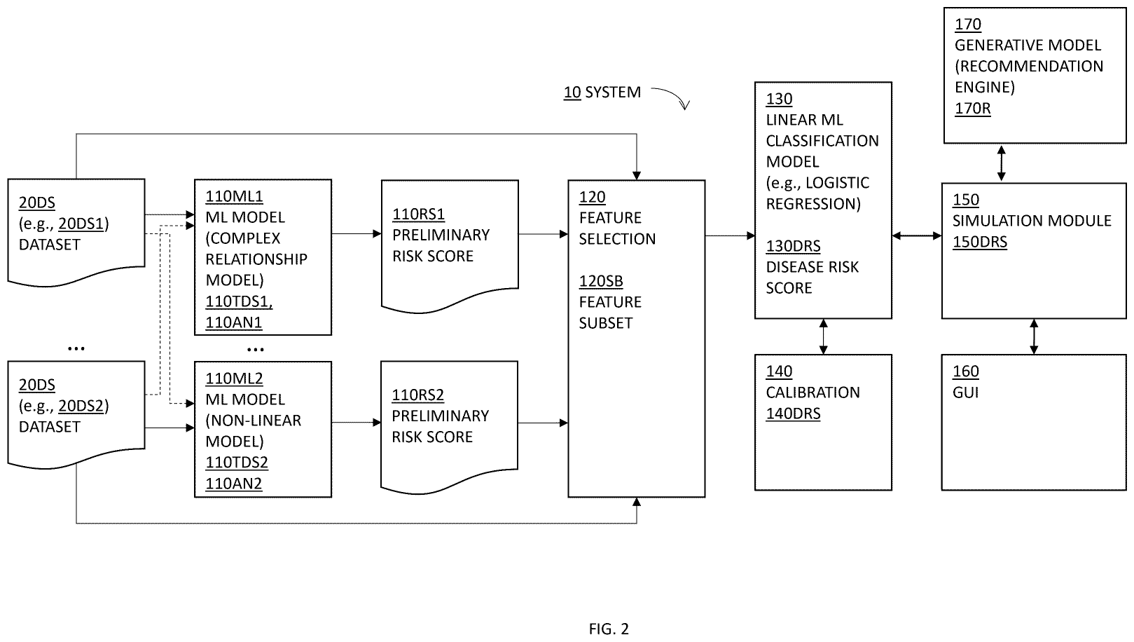

Reference is now made to FIG. 2, which depicts a system 10 for determining a disease risk score, according to some embodiments of the invention.

According to some embodiments of the invention, system 10 may be implemented as a software module, a hardware module, or any combination thereof. For example, system may be or may include a computing device such as element 1 of FIG. 1, and may be adapted to execute one or more modules of executable code (e.g., element 5 of FIG. 1) to determine a disease risk score of a subject, as further described herein.

As shown in FIG. 2, arrows may represent flow of one or more data elements to and from system 10 and/or among modules or elements of system 10. Some arrows have been omitted on FIG. 2 for the purpose of clarity.

According to some embodiments, system 10 may serve as a framework for predicting a disease risk score. As elaborated herein, system 10 may receive input data set(s) 20DS from multiple sources (e.g., 20DS1, 20DS2, etc.), process the data sets 20DS through different stages, and output a disease risk score 130DRS for a target subject.

Additionally, or alternatively, system 10 may provide a platform for perturbating data in input data set(s), and the effect of such perturbations on the subject's disease risk score 130DRS.

As shown in FIG. 2, system 10 may receive a first feature dataset 20DS1 and at least one second feature dataset 20DS2. Each feature dataset (e.g., 20DS1, 20DS2) may include values of one or more features that may each represent a property of the target subject.

For example, a first feature dataset 20DS1 may include values of genomic features, such as a Polygenic Risk Score (PRS), representing polygenic risk in manifesting a specific disease (e.g., a cardiovascular disease), monogenic data, representing traits of the target subject that are influenced by a single gene, pharmacogenomic data, representing the target subject's response to drugs, and the like.

In another example, at least one second dataset 20DS2 may include values of features pertaining to a first group of blood measurements, such as a blood pressure (BP) level, a total cholesterol level, a High Density Lipoprotein (HDL) level, a Low Density Lipoprotein (LDL) level, a Lipoprotein A level, and the like.

In another example, at least one second dataset 20DS2 may include values of features pertaining to a second group of blood measurements, such as a testosterone level, a C-reactive protein level, a basophil count, a cystatin-C level, a mean corpuscular hemoglobin value, and the like.

In yet another example, at least one second dataset 20DS2 may include values of features of the subjects, such as the subject's age, gender, ethnicity, prior diagnoses (e.g., a diabetes or asthma diagnosis), a status of drug treatment (e.g., anti-hypertensive treatment, statins-based treatment, and the like), a lifestyle factor (e.g., smoking habits, a usual walking pace), features related to mental health (e.g., experiencing loneliness or isolation, having mood swings, having seen a general-practitioner doctor for nervous behaviour, anxiety, tension, depression, etc.), and the like.

As shown in FIG. 2, system 10 may include a first non-linear Machine Learning (ML) based model 110ML1, adapted to handle features of the first feature dataset 20DS1. Additionally, or alternatively, system 10 may include at least one second non-linear ML based model 110ML2, adapted to respectively handle features of the at least one second feature dataset 20DS2.

According to some embodiments, first feature dataset 20DS1 and the at least one second feature dataset 20DS2 may overlap in one or more features. In such embodiments, specific features of dataset 20DS1 and dataset 20DS2 may be introduced in parallel as inputs to both first and second non-linear ML models.

Additionally, or alternatively, specific features may be missing from datasets 20DS1 or 20DS2 of specific subjects. In such embodiments, non-linear ML models 110ML1 and/or 110ML2 may employ a sparsity-aware algorithm, or imputing algorithm, to fill-in the missing data, as known in the art.

According to some embodiments, system 10 may be configured to apply the first non-linear ML based model on the first feature dataset 20DS1, to obtain a first preliminary risk score 110RS1. First preliminary risk score 110RS1 may represent a first assessed probability of the target subject in having, or manifesting a disease of interest.

The first non-linear ML model 110ML1 may include, for example, a Deep Learning Neural Network (DNN) model, having at least four neural layers. The first non-linear ML model 110ML1 may be trained as elaborated herein to predict an assessment of the subject as having a disease of interest (e.g., a cardiovascular disease) based on features (e.g., genomic data and other relevant features) of the first feature dataset 20DS1.

System 10 may receive (e.g., during a training stage, via input 7 of FIG. 1) a first training dataset 110TDS1. Training dataset 110TDS1 may include one or more (e.g., a plurality of) first, annotated feature datasets 20DS1, each pertaining to a respective subject of a first cohort of subjects. System 10 may further obtain (e.g., via input 7 of FIG. 1) a first set of annotations 110AN1, each labeling a condition of a corresponding subject of the first cohort of subjects.

Feature datasets 20DS1 of training dataset 110TDS1 may be annotated in a sense that they may be associated with respective annotations or labels 110AN1, which may indicate a condition of the respective subjects (e.g., a likelihood of subjects as manifesting the disease of interest).

As known in the art, system 10 may subsequently utilize a training scheme (e.g., a backward propagation scheme), to train non-linear ML model 110ML1, while using training dataset 110TDS1 as supervisory information. System 10 may thereby train the first non-linear ML model, so as to predict the first preliminary risk score 110RS1 of specific member subjects of the first cohort of subjects, based on the first training dataset 110TDS1.

In a subsequent, inference stage, non-linear ML model 110ML1 may be configured to receive a dataset 20DS1 representing features of a target subject or patient. Based on the training, non-linear ML model 110ML1 may predict, or produce a prediction of a preliminary risk score 110RS1, e.g., as either manifesting the disease of interest, or not.

Additionally, or alternatively, based on the training, non-linear ML model 110ML1 may produce a confidence value, that may represent a confidence of ML model 110ML1 in classifying dataset 20DS1 of a subject as either representing a healthy subject or a sick one. Non-linear ML model 110ML1 may subsequently produce preliminary risk score 110RS1 as a function of (e.g., equal to) the computed confidence value.

In other words, based on its training, non-linear ML model 110ML1 may map between features of a subject and a corresponding ranking value, that may represent a probability 110RS1 of that subject as having the disease of interest.

During an inference stage, system 10 may infer ML model 110ML1 on dataset 20DS1 of a specific target subject, to obtain a preliminary risk score 110RS1 corresponding to the target subject.

Additionally, or alternatively, ML model 110ML2 may be another non-linear machine learning model applied to the at least one second feature dataset 20DS2 to obtain at least one respective second preliminary risk score 110RS2.

The second non-linear ML model 110ML2 may include, for example, a decision tree model, a K-Nearest Neighbors model, a non-linear Support Vector Machine (SVM) model, or a Naive Bayes model with non-linear transformations.

The at least one second, non-linear ML model 110ML2 may be trained as elaborated herein to predict the at least one respective second preliminary risk score 110RS2 as a second assessed probability of the target subject in manifesting the disease of interest, based on values of features (e.g., blood measurements, age, gender, ethnicity, prior diagnoses, status of drug treatment, lifestyle features, mental health-related features, etc.) of the at least one second feature dataset 20DS2.

System 10 may receive (e.g., during a training stage, via input 7 of FIG. 1) a second training dataset 110TDS2. Training dataset 110TDS2 may include one or more (e.g., a plurality of) second, annotated feature datasets 20DS2, each pertaining to a respective subject of a second cohort of subjects (which may, or may not overlap with the first cohort of subjects).

System 10 may further obtain (e.g., via input 7 of FIG. 1) a second set of annotations 110AN2, each labeling a condition of a corresponding subject of the second cohort of subjects.

Feature datasets 20DS2 of training dataset 110TDS2 may be annotated in a sense that they may be associated with respective annotations or labels 110AN2, which may indicate a condition of the respective subjects (e.g., a likelihood of subjects as manifesting the disease of interest).

As known in the art, system 10 may subsequently utilize a training scheme (e.g., a backward propagation scheme), to train non-linear ML model 110ML2, while using training dataset 110TDS2 as supervisory information. System 10 may thereby train the second non-linear ML model, so as to predict the second preliminary risk score 110RS2 of specific member subjects of the second cohort of subjects, based on the second training dataset 110TDS2.

In a subsequent, inference stage, the at least one non-linear ML model 110ML2 may be configured to receive at least one respective dataset 20DS2 representing features of a target subject or patient. Based on the training, non-linear ML model 110ML2 may predict, or produce a prediction of a preliminary risk score 110RS2, e.g., either manifesting the disease of interest, or not.

Additionally, or alternatively, based on the training, non-linear ML model 110ML2 may produce a confidence value, that may represent a confidence of ML model 110ML2 in classifying a dataset 20DS2 of a subject as either representing a healthy subject or a sick one. At least one non-linear ML model 110ML2 may subsequently produce preliminary risk score 110RS2 as a function of (e.g., equal to) the computed confidence value.

In other words, based on its training, at least one non-linear ML model 110ML2 may map between features of a subject and a corresponding ranking value, that may represent a probability 110RS2 of that subject as having the disease of interest.

During an inference stage, system 10 may infer ML model 110ML2 on dataset 20DS2 of a specific target subject, to obtain at least one preliminary risk score 110RS2 corresponding to the target subject.

As shown in FIG. 2, system 10 may include a feature selection module 120. Feature selection module 120 may be adapted to apply a feature selection algorithm, to identify a subset 120SB of features from the first feature dataset 20DS1 and at least one second feature dataset 20DS2, as prominent contributors in predicting the preliminary risk scores 110RS1, 110RS2.

According to some embodiments, feature selection module 120 may apply a feature selection algorithm, to identify a first group of features from the first feature dataset 20DS1, as prominent contributors (e.g., beyond a predefined threshold) in predicting the first preliminary risk score 110RS1, and apply the feature selection algorithm, to identify a second group of features from the second feature dataset 20DS2, as prominent contributors (e.g., beyond the predefined threshold) in predicting the second preliminary risk score 110RS2. Feature selection module 120 may subsequently select the subset of features 120SB of the plurality of features based on the first, and second groups of features. For example, features may be selected to be included in the feature subset 120SB when their P value is below a predetermined value (e.g., 0.05), when forward selection is applied.

As shown in FIG. 2, system 10 may further include a linear, ML-based classification or regression model 130, such as a logistic regression model. Linear model 130 may be configured to apply a linear machine learning-based function on (i) the first preliminary risk score 110RS1, (ii) the second preliminary risk score 110RS2, and (iii) the subset of features from the meta-dataset 120MDS, to determine an overall disease risk score 130DRS. Disease risk score 130DRS may represent an overall probability of the target subject in having, or manifesting the disease of interest.

According to some embodiments, system 10 may (e.g., during a training stage) selecting a subset 120SB of features from the first dataset 20DS1 and/or second dataset 20DS2, pertaining to a specific subject of the first cohort and/or second cohort. System 10 may use at least one of (i) the first set of annotations 110AN1 and (ii) the second set of annotations 110AN2 as supervisory data, to train linear ML-based model 130, so as to produce an initial prediction, or initial value of disease risk score 130DRS of the specific subject.

It may be appreciated that, as ML-based model 130 is a linear model, the initial prediction 130DRS may be a linear combination of the inputs to ML-based model 130. In other words, initial prediction 130DRS may be a linear combination of the subset of features 120SB pertaining to the specific subject, and (a) the first preliminary risk score 110RS1 of the specific subject and/or (b) the at least one second preliminary risk score 110RS2 of the specific subject.

As shown in FIG. 2, system 10 may further include a calibration module 140, adapted to fine-tune the initial value of the disease risk score 130DRS, based on disease-specific statistical properties.

For example, The calibration module 140 may calculate statistical properties characterizing the manifestation of the disease in a population of the first cohort and/or second cohort, based on at least one of the first and second sets of annotations. For example, calibration module 140 may calculate a mean and standard deviation of manifestation of the disease in population groups that are defined by gender, age, ethnicity, and the like.

calibration module 140 may subsequently obtain, from the linear ML-based model, an initial value of the disease risk score 130DRS for a specific target subject, and fine-tune, or calibrate the initial value of the disease risk score 130DRS based on the disease-specific statistical properties, to determine a revised, or amended disease risk score 140DRS of the target subject.

As elaborated herein, the combination of linear, and non-linear models may allow system 10 address two, seemingly contradicting objectives: On one hand, the employment of complex, non-linear models (110ML1, 110ML2) in an ensemble-learning architecture allows system 10 to learn complex and non-linear relations between features characterizing a variety of patients, and their respective likelihood of manifesting a disease of interest. On the other hand, the employment of a linear model (130) allows system 10 to provide an explainable, intuitive, and insightful interface, for understanding the contributors to a subjects condition, as explained below.

As shown in FIG. 2, system 10 may further include a simulation module 150 and a Graphic User Interface (GUI) module 160 (e.g., input 7 and output 8 of FIG. 1).

According to some embodiments, GUI 160 may provide an interface for users to interact with system 10. For example, GUI 160 may allow users to input data, manipulate risk factor values (e.g., features of subset 120SB), and visualize the effect of changes on the disease risk score. GUI 160 may also present disease risk score 130DRS/140DRS and treatment recommendations.

Simulation module 150 may collaborate with GUI 160 to allow perturbation of feature values, so as to simulate different “what if” scenarios, and their impact on disease risk score 130DRS/140DRS.

In some embodiments, simulation module 150 may receive perturbations or changes in feature values via GUI 160, and apply the linear ML-based model on the changed feature values to determine simulated disease risk scores 130DRS/140DRS. Simulation module 150 may subsequently present the simulated disease risk scores 130DRS/140DRS via the GUI 160.

Additionally, or alternatively, system 10 may include a generative model 170, also referred to herein as a “recommendation engine” 170. Generative model 170 may be configured to collaborate with simulation module 150 so as to automatically perturb feature values of feature subset 120SB. Generative model 170 may thereby generate recommendations 170R for diminishing the target subject's probability of manifesting the disease. Generative model 170 may apply the linear ML-based model to the perturbed feature values, and collaborate with GUI 160, to present the simulated disease risk scores via the GUI 160 as recommendations for personalized healthcare.

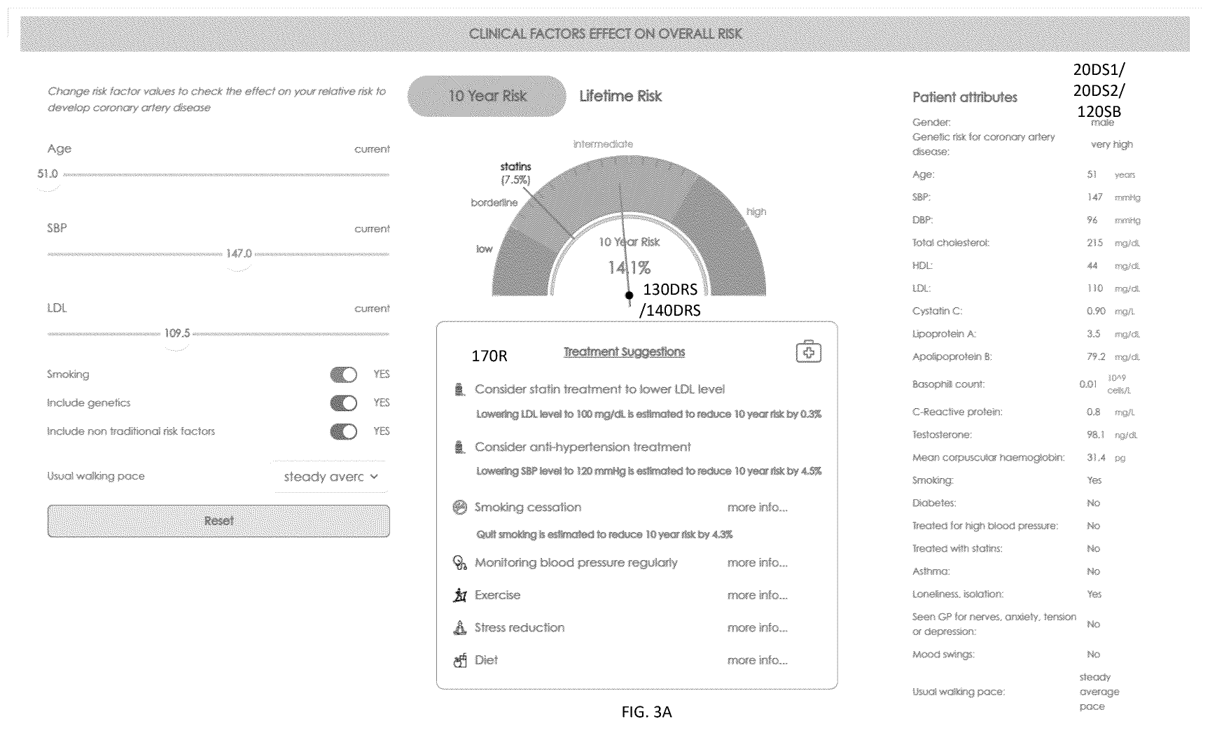

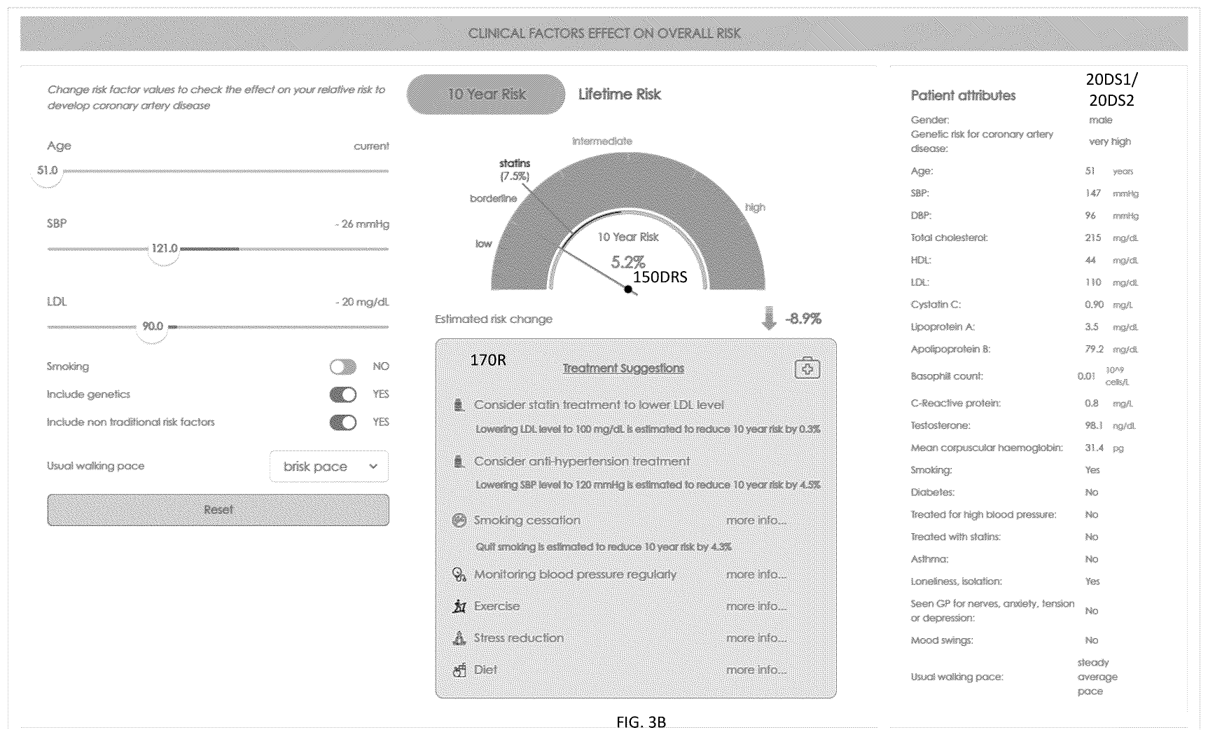

Reference is further made to FIGS. 3A and 3B, which are screenshots of GUI 160, according to some embodiments of the invention. As shown in FIG. 3A, system 10 may present a calculated value of a disease risk score 130DRS/140DRS (here—14.1%), based on the given patient feature datasets 20DS (denoted “Patient attributes”). The disease risk scores 130DRS/140DRS in FIG. 3A represent a risk of manifesting the disease (e.g., a cardiovascular disease) within a predefined time period (e.g., 10 years). GUI 160 may alternatively present a predicted disease risk scores 130DRS/140DRS pertaining to the subject's entire expected lifetime.

As shown in FIG. 3A, GUI 160 may further present recommendations 170R for changing a lifestyle (e.g., exercise, diet, stress reduction, etc.) and/or treatment, such as a statins-based treatment. In this example, the statins-based treatment is predicted to improve (reduce) the predicted disease risk score 130DRS/140DRS to 7.5%.

As shown in FIG. 3B, GUI 160 may further allow introduction of perturbations to some characteristic features of the subject, including, for example, changing their LDL level, and smoking habits. GUI 160 may subsequently present a simulated (“what if”) result 150DRS of the disease risk score (here—5.2%), representing the effect of those changes or perturbations.

Provide a recommendation of treatment (e.g., losing weight), alongside an expected improvement in the calculated disease risk score, as a result of that treatment.

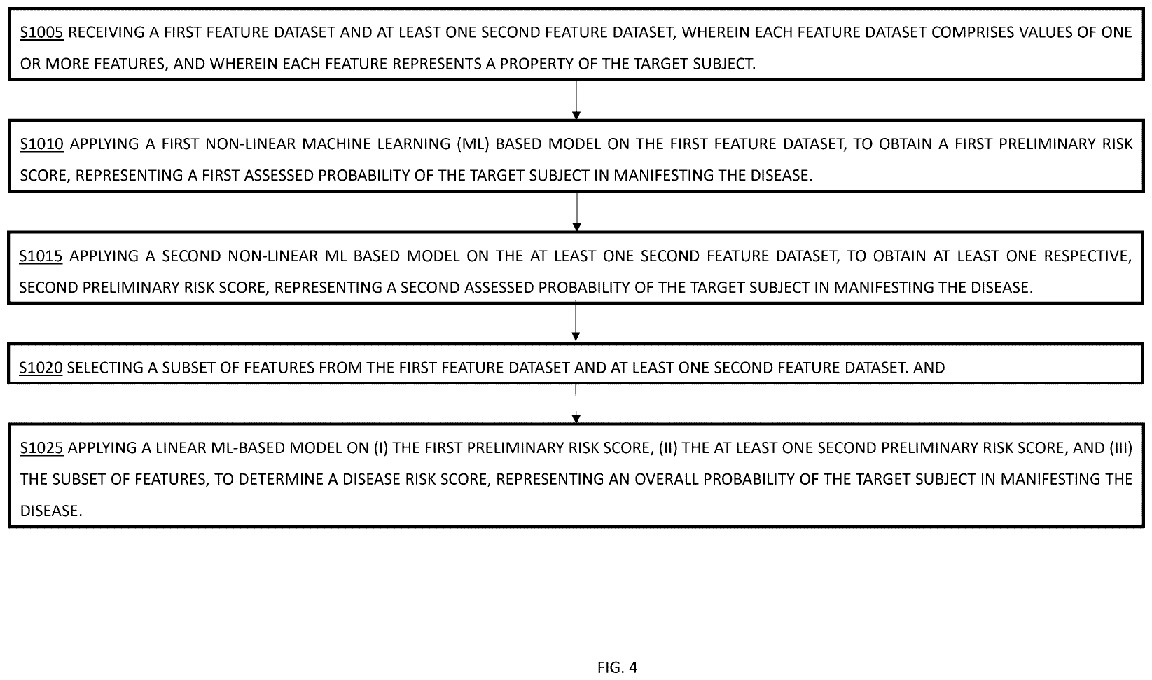

Reference is now made to FIG. 4, which is a flow diagram, depicting steps of a method of determining a disease risk score by at least one processor (e.g., processor 2 of FIG. 1), according to some embodiments of the invention.

As shown in step S1005, the at least one processor 2 may receive a first feature dataset 20DS1 and at least one second feature dataset 20DS1, where each feature dataset includes values of one or more features or attributes, and wherein each feature represents a property of the target subject.

As shown in step S1010, the at least one processor 2 may apply a first non-linear ML based model (e.g., element 110ML1 of FIG. 2) on the first feature dataset 20DS1, to obtain a first preliminary risk score (e.g., element 110RS1 of FIG. 2), representing a first assessed probability of the target subject in manifesting the disease.

As shown in step S1015, the at least one processor 2 may apply a second non-linear ML based model (e.g., element 110ML1 of FIG. 2) on the at least one second feature dataset 20DS2, to obtain at least one respective, second preliminary risk score (e.g., element 110ML2 of FIG. 2), representing a second assessed probability of the target subject in manifesting the disease.

As shown in step S1020, the at least one processor 2 may select (e.g., by feature selection module 120 of FIG. 2) a subset of features (120SB) from the first feature dataset 20DS1 and at least one second feature dataset 20DS2.

As shown in step S1025, the at least one processor 2 may subsequently apply a linear ML-based model (e.g., element 130 of FIG. 2) on (i) the first preliminary risk score 110RS1, (ii) the at least one second preliminary risk score 110RS2, and (iii) the subset of features 120SB, to determine a disease risk score (e.g., elements 130DRS/140DRS/150DRS of FIG. 2), representing an overall probability of the target subject in manifesting the disease.

Embodiments of the invention include a practical application for improving a technology of assistive diagnostics and disease risk assessment. Unless explicitly stated, the method embodiments described herein are not constrained to a particular order or sequence. Furthermore, all formulas described herein are intended as examples only and other or different formulas may be used. Additionally, some of the described method embodiments or elements thereof may occur or be performed at the same point in time.

While certain features of the invention have been illustrated and described herein, many modifications, substitutions, changes, and equivalents may occur to those skilled in the art. It is, therefore, to be understood that the appended claims are intended to cover all such modifications and changes as fall within the true spirit of the invention.

Various embodiments have been presented. Each of these embodiments may of course include features from other embodiments presented, and embodiments not specifically described may include various features described herein.

1. A method of predicting, by at least one processor, a probability of a target subject in manifesting a disease, the method comprising:
receiving a first feature dataset and at least one second feature dataset, wherein each feature dataset comprises values of one or more features, and wherein each feature represents a property of the target subject;
applying a first non-linear Machine Learning (ML) based model on the first feature dataset, to obtain a first preliminary risk score, representing a first assessed probability of the target subject in manifesting the disease;
applying a second non-linear ML based model on the at least one second feature dataset, to obtain at least one respective, second preliminary risk score, representing a second assessed probability of the target subject in manifesting the disease;
selecting a subset of features from the first feature dataset and at least one second feature dataset; and
applying a linear ML-based model on (i) the first preliminary risk score, (ii) the at least one second preliminary risk score, and (iii) the subset of features, to determine a disease risk score, representing an overall probability of the target subject in manifesting the disease.

2. The method of claim 1, wherein the first feature dataset and the at least one second feature dataset overlap in one or more features.

3. The method of claim 1, wherein the first non-linear ML model comprises a Deep learning Neural Network (DNN), having at least 4 neural layers.

4. The method of claim 3, wherein the first feature dataset comprises values of genomic features, selected from a list consisting of: a Polygenic Risk Score (PRS), representing polygenic risk in manifesting the disease, monogenic data, representing traits of the target subject that are influenced by a single gene, and pharmacogenomic data, representing the target subject's response to drugs.

5. The method of claim 1 wherein the at least one second non-linear ML model is selected from a list consisting of a decision tree model, a k-Nearest Neighbors model, a non-linear Support Vector Machine (SVM) model, and a Naive Bayes model, having non-linear transformations.

6. The method of claim 5, wherein the at least one second feature dataset comprises values of features pertaining to a first group of blood measurements, selected from: a blood pressure (BP) level, a total cholesterol level, a High Density Lipoprotein (HDL) level, a Low Density Lipoprotein (LDL) level, a Lipoprotein A level.

7. The method of claim 6, wherein the at least one second feature dataset comprises values of features pertaining to a second group of blood measurements, selected from: a testosterone level, a C-reactive protein level, a basophil count, a cystatin-C level, and a mean corpuscular hemoglobin value.

8. The method of claim 5, wherein the at least one second feature dataset comprises values of features of the subject, selected from a list consisting of: an age, a gender, an ethnicity, a prior diagnosis, a status of drug treatment, a lifestyle factor, and a feature related to mental health.

9. The method of claim 1, further comprising:
receiving a first training dataset, comprising a plurality of first feature datasets, each pertaining to a respective subject of a first cohort of subjects;
obtaining a first set of annotations, each labeling a condition of a corresponding subject of the first cohort of subjects; and
using the first set of annotations as supervisory data, to train the first non-linear ML model, so as to predict the first preliminary risk score of subjects of the first cohort of subjects, based on the first training dataset.

10. The method of claim 9, further comprising:
receiving a second training dataset, comprising a plurality of second feature datasets, each pertaining to a respective subject of a second cohort of subjects;
obtaining a second set of annotations, each labeling a condition of a corresponding subject of the second cohort of subjects; and
using the second set of annotations as supervisory data, to train the second non-linear ML model, so as to predict the second preliminary risk score of subjects of the second cohort of subjects, based on the second training dataset.

11. The method of claim 10, further comprising:
selecting the subset of features from the first dataset and/or second dataset, wherein the subset of features pertains to a specific subject of the first cohort and/or second cohort; and
using at least one of the (i) first set of annotations and (ii) second set of annotations as supervisory data, to train the linear ML-based model to produce an initial prediction of a disease risk score of the specific subject,
wherein said initial prediction is a linear combination of the subset of features of the specific subject, and (a) the first preliminary risk score of the specific subject and/or (b) the at least one second preliminary risk score of the specific subject.

12. The method of claim 1, further comprising:
applying a feature selection algorithm, to identify a first group of features from the first feature dataset, as prominent contributors in predicting the first preliminary risk score;
applying the feature selection algorithm, to identify a second group of features from the second feature dataset, as prominent contributors in predicting the second preliminary risk score; and
selecting the subset of features of the plurality of features based on the first, and second groups of features.

13. The method of claim 10, further comprising:
calculating one or more disease-specific statistical properties, characterizing manifestation of the disease in a population of the first cohort and/or second cohort, based on at least one of the first and second sets of annotations;
obtaining, from the linear ML-based model, an initial value of the disease risk score for the target subject; and
fine-tuning the initial value of the disease risk score, based on the disease-specific statistical properties, to determine the disease risk score of the target subject.

14. The method of claim 1 further comprising
receiving, via a Graphical User Interface (GUI) a perturbation of a value of at least one feature of the subset of features;
applying the linear ML-based model on the subset of features having the perturbed feature value, to determine a simulated disease risk score, representing a simulated probability of the target subject in manifesting the disease; and
presenting the simulated disease risk score via the GUI as a result of said perturbation.

15. The method of claim 1 further comprising
automatically perturbing a value of one or more features of the subset of features;
applying the linear ML-based model on the subsets of features, each having at least one perturbed feature value, to determine corresponding simulated disease risk scores, wherein each simulated disease risk score represents a simulated probability of the target subject in manifesting the disease, as a result of the corresponding at least one perturbation; and
presenting the simulated disease risk scores via a GUI, as recommendations for diminishing the target subject's probability of manifesting the disease.

16. A system for predicting a disease risk score of a subject, the system comprising: a non-transitory memory device, wherein modules of instruction code are stored, and at least one processor associated with the memory device, and configured to execute the modules of instruction code, whereupon execution of said modules of instruction code, the at least one processor is configured to:
receive a first feature dataset and at least one second feature dataset, wherein each feature dataset comprises values of one or more features, and wherein each feature represents a property of the target subject;
apply a first non-linear Machine Learning (ML) based model on the first feature dataset, to obtain a first preliminary risk score, representing a first assessed probability of the target subject in manifesting the disease;
apply a second non-linear ML based model on the at least one second feature dataset, to obtain at least one respective, second preliminary risk score, representing a second assessed probability of the target subject in manifesting the disease;
select a subset of features from the first feature dataset and at least one second feature dataset; and
apply a linear ML-based model on (i) the first preliminary risk score, (ii) the at least one second preliminary risk score, and (iii) the subset of features, to determine a disease risk score, representing an overall probability of the target subject in manifesting the disease.

17. The system of claim 16, wherein the at least one processor is further configured to:
apply a feature selection algorithm, to identify a first group of features from the first feature dataset, as prominent contributors in predicting the first preliminary risk score;
apply the feature selection algorithm, to identify a second group of features from the second feature dataset, as prominent contributors in predicting the second preliminary risk score; and
select the subset of features of the plurality of features based on the first, and second groups of features.

18. The system of claim 16, wherein the at least one processor is further configured to:
receive, via a GUI, a perturbation of a value of at least one feature of the subset of features;
apply the linear ML-based model on the subset of features having the perturbed feature value, to determine a simulated disease risk score, representing a simulated probability of the target subject in manifesting the disease; and
present the simulated disease risk score via the GUI as a result of said perturbation.

19. The system of claim 16, wherein the at least one processor is further configured to:
automatically perturb a value of one or more features of the subset of features;
applying the linear ML-based model on the subsets of features, each having at least one perturbed feature value, to determine corresponding simulated disease risk scores, wherein each simulated disease risk score represents a simulated probability of the target subject in manifesting the disease, as a result of the corresponding at least one perturbation; and
presenting the simulated disease risk scores via a GUI, as recommendations for diminishing the target subject's probability of manifesting the disease.
